(19) United States
(12) Patent Application Publication
(10) Pub. No.: US 2021/0225512 A1
(43) Pub. Date: Jul. 22, 2021

(54) DIAGNOSIS SUPPORT SYSTEM

(71) Applicant: IRYOU JYOUHOU GIJYUTU KENKYUSHO CORPORATION, Fukuoka (JP)

(72) Inventor: Shinkichi HIMENO, Fukuoka (JP)

(73) Assignee: IRYOU JYOUHOU GIJYUTU KENKYUSHO CORPORATION, Fukuoka (JP)

(21) Appl. No.: 17/059,950

(22) PCT Filed: Jul. 16, 2019
(86) PCT No.: PCT/JP2019/027900

§ 371 Date: Nov. 30, 2020

(30) Foreign Application Priority Data
Jul. 26, 2018 (JP) 2018-140535

(51) Int. Cl. G16H 50/20; G16H 10/60; G16H 70/60; G16H 50/70; G16H 40/20; G06F 16/245

(52) U.S. Cl. CPC G16H 50/20; G16H 10/60; G06F 16/245; G16H 50/70; G16H 40/20; G16H 70/60

(57) ABSTRACT

[Problem] In regard to narrowed-down disease name groups, to determine a combination of disease names having a big frequency difference therebetween on the basis of aggregation frequency of case lists for each symptom/finding or symptom/finding pair of respective diseases using the case list and to recommend an examination and an inspection using the determined combination of disease names as symptoms/findings or symptom/finding pairs to be obtained next for confirming a diagnostic name.

[Solution] The present invention comprises: a symptom/finding management means for recording and managing symptoms and inspection findings of a patient to which diagnosis has been confirmed; a disease-name management means for recording and managing lists of cases corresponding to the disease and symptoms and findings observed in such cases for each confirmed disease name; a disease-name confirmed-case registration means for registering disease names of cases, the disease name of which has been confirmed, and symptoms and inspection findings observed in the cases; and a disease-name unconfirmed-case inquiring means for presenting symptoms and inspection findings of disease-name unconfirmed cases to the disease-name confirmed-case management means and inquiring highly-possible disease names and symptoms and inspection findings effective for confirming a disease name to be obtained next.

### TECHNICAL FIELD

The present invention relates to a diagnosis support system that contributes to efficient and less overlooked medical care by registration of a large number of cases whose diagnosis names have been confirmed in the past, and by recommendation, for cases whose disease names are currently unconfirmed, on a list of disease names that are inferred and medical examinations and tests that are the “next move” that are useful for advancing the differentiation of the disease name, on the basis of frequency of disease names and symptoms.

### BACKGROUND ART

Many patients visit medical institutions every day for medical examinations and tests, and when disease names are confirmed, they receive corresponding treatment. A doctor guesses the disease name from the experience of the cases experienced in the past, and comprehensively summarizes the examination findings (hereinafter, symptoms/findings) obtained from the symptoms obtained by interviews and physical findings, the imaging findings obtained by blood tests and Xp, and the like to narrow down the candidates for the disease name. The experience and intuition of an experienced doctor is great, but unfortunately that experience and intuition is lost in the death of the doctor.

Attempts have been made to accumulate past cases in computers and the like, obtain the frequency and correlation of disease names and symptoms/findings, and use them to support diagnosis. For example, there are Patent Literatures 1 and 2. In addition, as in Non-Patent Literature 1, there is also an attempt to estimate candidate disease names from observed symptoms/findings using Bayesian probabilities.

### CITATION LIST

### Patent Literature

Patent Literature 1: JP 2003-108665 A
Patent Literature 2: JP 2000-298696 A

### Non Patent Literature

Non Patent Literature 1: https://www.kantei.go.jp/jp/singi/keizaisaisei/miraitoshikaigi/suishinkaigo_iryokaigo_dai2/siryou5.pdf

### SUMMARY OF INVENTION

### Technical Problem

When diagnosing a disease name, a doctor guesses the disease name from the relationship between the combination of symptoms/findings and the disease name, and the disease name is narrowed down by adding the symptoms/findings while considering the frequency of the disease (differential diagnosis).

In each of the above literatures, it is argued that the disease names are narrowed down from the frequency distribution of the disease name and the frequency of symptoms/findings and the partial symptom/finding group of the currently obtained cases with unconfirmed disease names.

However, in the above literatures, it is hard to say that the database structure, narrowing down, recommendation logic, and the like are clearly explained and means therefor are disclosed in regard to the database structure of past diagnosis confirmed cases, how to systematically obtain the frequency distribution, how to narrow down the disease names using Bayesian probabilities, what kind of findings/tests are useful for further advance of the diagnosis.

Regarding the frequency distribution of disease names and symptoms/findings, the number of applicable cases is added one by one and included in a summary table. Therefore, it is not possible to perform a detailed analysis returning to the original case that constitutes the frequency distribution.

In addition, the frequency distribution is limited to distribution for each symptom/finding, and the frequency distribution for each symptom/finding pair is not considered. Some symptoms/findings are strongly correlated with each other. In this case, the symptoms/findings with strong correlation merely represent similar information duplicately, and if they are used as it is when calculating Bayesian probabilities or the like, there is a risk of overestimating the probability of the disease.

For each of disease names and symptoms/findings, a tree structure consisting of superordinate concepts/subordinate concepts is adopted. The lower the concept, the more information there is, but the information may be dispersed and the number of cases is small and not sufficient for analysis.

Since the target diseases differ depending on the medical institution, the disease name and the frequency of symptoms/findings differ greatly. For example, if there is a complaint of chest pain at a hospital specializing in heart disease, myocardial infarction is to be suspected first, but in orthopedics, there is a high possibility of a rib fracture. As described above, the difference in frequency distribution depending on the attributes such as medical institutions and wards should be taken into consideration.

When the contents such as the case list accumulated in the disease name confirmed case management means become enormous, it tends to be difficult to confirm mutual consistency and grasp the support logic.

The present invention has been made to solve such a conventional problem, and an object of the present invention is to provide a disease name confirmed case management means that, by a disease name confirmed case registration means, facilitates a case list having a symptom/finding or a symptom/finding pair for each disease name, and a case list—disease name list for each symptom/finding or symptom/finding pair, for past cases of which disease name has been confirmed. An object of the present invention is to provide a similar case search means that searches for similar cases having symptoms/findings or symptom/finding pair already observed in diagnosis name unconfirmed cases by utilizing various case lists accumulated in the disease name confirmed case management means, and to narrow down candidates for disease names by sequentially calculating the probability of disease names using Bayesian probabilities or the like using disease name frequency distribution for each symptom/finding or symptom/finding pair that has already been observed.

An object of the present invention is to, regarding the narrowed down disease name group, obtain the one with a large frequency difference among disease names from the aggregation frequency of the case list for each symptom/finding or symptom/finding pair of each disease name to recommend a medical examination or test with the one as the symptom/finding or symptom/finding pair to be obtained next for diagnosis name confirmation.

Furthermore, an object of the present invention is to reduce the evaluation of the symptom/finding or symptom/finding pair that is highly correlated with the symptom/findings or symptom/finding pairs that have already been observed when making the above recommendation, and adjust the degree of recommendation for the medical examination or test that requires labors and costs. In addition, for the disease names and symptoms/findings, a tree-like master-slave relationship having a superordinate concept and a subordinate concept is adopted. An object of the present invention is to secure a sufficient case list by integrating the case list group of the subordinate concept into the case list of the superordinate concept as needed. An object of the present invention is to enable setting of a scope of search and enable inquiry using a group of diagnosis name confirmed cases suitable for diagnosis name unconfirmed cases, since there is a large bias in the target diseases of medical institutions.

An object of the present invention is to export/import a part or all of the contents accumulated by the disease name confirmed case management means to enable utilization of the accumulated contents of not only the own hospital but also other medical institutions. If the contents such as the case list accumulated in the disease name confirmed case management means can be viewed, not only the confirmation of the consistency of the contents but also the viewing itself is useful for understanding the pathological condition.

### Solution to Problem

As a means for achieving the above-mentioned object, a diagnosis support system according to claim 1 includes a disease name confirmed case management means including (i) a symptom/finding management means that records and manages a symptom and an examination finding of a patient whose diagnosis has been confirmed, (ii) a disease name management means that records and manages, for each disease name that has been confirmed, a list of cases corresponding to a corresponding disease, and a symptom and a finding observed in a corresponding case, and (iii) a disease name confirmed case registration means that registers a disease name of a case of which disease name has been confirmed, and a symptom and an examination finding observed in a corresponding case in (i) and (ii),

in which the diagnosis support system further includes a disease name unconfirmed case inquiry means that, to the disease name confirmed case management means, presents a symptom and an examination finding of a disease name unconfirmed case to inquire about a disease name having high possibility, a symptom and an examination finding to be obtained next which is effective for confirming a disease name.

In the diagnosis support system according to claim 2, the diagnosis support system according to claim 1 includes, in the symptom/finding management means, a case list management means for each symptom/finding that manages a list of cases in which a corresponding symptom/finding is observed for each symptom/finding.

In the diagnosis support system according to claim 3, the diagnosis support system according to claim 1 or 2 includes, in the symptom/finding management means, a disease name list management means for each symptom/finding that manages a list of disease names for each of a corresponding list of cases for each symptom/finding.

In the diagnosis support system according to claim 4, the diagnosis support system according to any one of claims 1 to 3 includes, in the case list management means for each symptom/finding, a case list management means for each symptom/finding pair that manages a list of cases for each symptom/finding pair observed simultaneously in each case.

In the diagnosis support system according to claim 5, the diagnosis support system according to any one of claims 1 to 4 includes, in the disease name management means, a case list management means for each symptom/finding for each disease name that manages a list of cases that have been observed for each symptom/finding that has been observed in cases corresponding to the disease name.

In the diagnosis support system according to claim 6, the diagnosis support system according to claim 5 includes, in the case list management means for each symptom/finding for each disease name, a case list management means for each symptom/finding pair for each disease name that manages a list of cases for each symptom/finding pair observed simultaneously in each case corresponding to a corresponding disease name.

In the diagnosis support system according to claim 7, the diagnosis support system according to any one of claims 1 to 6 includes, in the disease name management means, a case list management means for each disease name that manages a list of cases corresponding to the disease.

In the diagnosis support system according to claim 8, the diagnosis support system according to any one of claims 1 to 7 includes, in the disease name confirmed case management means, a disease name confirmed case data management means that records/manages a list of a disease name of a case registered in the disease name confirmed case registration means and a symptom/finding that has been observed.

In the diagnosis support system according to claim 9, the diagnosis support system according to any one of claims 1 to 8 includes, in the disease name unconfirmed case inquiry means, at least one of: a similar case search means that obtains, for each symptom/finding observed in the disease name unconfirmed case, a product set of a list of cases having a corresponding symptom/finding to display case data of corresponding case; and a similar case disease name estimation means that totals confirmed disease names of cases obtained by the product set to display the confirmed disease names in order of frequency.

In the diagnosis support system according to claim 10, the diagnosis support system according to any one of claims 1 to 9 includes, in the disease name unconfirmed case inquiry means, a disease name candidate list estimation means that estimates a disease name candidate list by using (i) frequency distribution of a confirmed disease name obtained by the disease name list management means for each symptom/finding for each symptom/finding, the frequency distribution being observed in a corresponding disease name unconfirmed case, (ii) frequency distribution of a confirmed disease name of a case list obtained by the case list management means for each symptom/finding pair for each symptom/finding pair, the frequency distribution being observed in a corresponding disease name unconfirmed case, or using both of (i) and (ii).

In the diagnosis support system according to claim 11, the diagnosis support system according to claim 9 or 10 includes, in the disease name unconfirmed case inquiry means, a medical examination/test recommendation means that compares aggregation frequencies of a case list for each symptom finding or symptom finding pair obtained by the case list management means for each symptom/finding for each disease name or the case list management means for each symptom/finding pair for each disease name, for each of disease name candidates obtained by one of a confirmed disease name frequency order list obtained by the similar case disease name estimation means and a disease name candidate list obtained by the disease name candidate list estimation means, to recommend a symptom/finding or symptom/finding pair with a large difference in aggregation frequency among the disease name candidates as a symptom/finding or symptom/finding pair to be obtained next.

In the diagnosis support system according to claim 12, the diagnosis support system according to any one of claims 1 to 11 includes, in the medical examination/test recommendation means, a recommendation degree reduction means, using correlation, that determines a symptom/finding that has not been performed and are highly correlated with a symptom/finding that has already been recommended and performed by frequency distribution of confirmed disease names of a case list obtained by the case list management means for each symptom/finding pair to reduce a recommendation degree of the symptom/finding that has not been performed and are highly correlated.

In the diagnosis support system according to claim 13, the diagnosis support system according to any one of claims 1 to 12 includes, in the medical examination/test recommendation means, a cost corresponding medical examination and test recommendation means that adjusts a recommendation priority by time and money costs required for execution.

In the diagnosis support system according to claim 14, the diagnosis support system according to any one of claims 1 to 13 includes, in the symptom/finding management means, a symptom/finding case link group integration means that describes an inclusion relationship between individual symptom/finding as a master-slave relationship link, and integrates a case link group recorded in a slave symptom/finding to a master case link group as necessary.

In the diagnosis support system according to claim 15, the diagnosis support system according to any one of claims 1 to 14 includes, in the disease name management means, a disease name case link group integration means that describes an inclusion relationship between individual disease names as a master-slave relationship link, and integrates a case link group recorded in a slave disease name to a master disease name case link group as necessary.

In the diagnosis support system according to claim 16, the diagnosis support system according to any one of claims 1 to 15 includes, in the disease name confirmed case registration means, an electronic medical record direct registration means that enables direct registration of a disease name and symptom/finding of a case of which disease name has been confirmed, from an electronic medical record of a corresponding patient.

In the diagnosis support system according to claim 17, the diagnosis support system according to any one of claims 1 to 16 includes, in the disease name unconfirmed case inquiry means, an electronic medical record direct inquiry means that enables direct inquiry of a symptom/finding of a case of which disease name has not been confirmed, from an electronic medical record of a corresponding patient to the disease name confirmed case management means.

In the diagnosis support system according to claim 18, the diagnosis support system according to any one of claims 1 to 17 includes, in the disease name confirmed case registration means, a case attribute management means that performs management by imparting a case attribute such as a medical institution in which a case to be registered occurs, medical department, doctor in charge, and age, gender, and race of a patient.

In the diagnosis support system according to claim 19, the diagnosis support system according to any one of claims 1 to 18 includes, in the disease name unconfirmed case inquiry means, a search scope management means that sets, as a search target, only a case having a specific case attribute among disease name confirmed cases managed by the disease name confirmed case management means.

In the diagnosis support system according to claim 20, the diagnosis support system according to any one of claims 1 to 19 includes a disease name confirmed case exporting means that exports a part or all of contents recorded in the symptom/finding management means, the disease name management means, and the disease name confirmed case registration means included in the disease name confirmed case management means, to another disease name confirmed case management means.

In the diagnosis support system according to claim 21, the diagnosis support system according to claim 20 includes a disease name confirmed case importing means that imports contents extracted by the disease name confirmed case exporting means to reconstruct contents of the symptom/finding management means, the disease name management means, and the disease name confirmed case registration means.

In the diagnosis support system according to claim 22, the diagnosis support system according to any one of claims 1 to 21 includes, in the disease name confirmed case management means, a disease name confirmed case management means content viewing means that views a management content of the symptom/finding management means, the disease name management means, and the disease name confirmed case registration means.

### Advantageous Effects of Invention

Since the diagnosis support system according to claim 1 includes the symptom/finding management means, it is possible to record and manage the symptom and the examination findings of a patient whose diagnosis has been confirmed.

Since the diagnosis support system includes the disease name management means, it is possible to record and manage a list of cases corresponding to the disease and symptoms and findings observed in the cases for each confirmed disease name.

Since the diagnosis support system includes the disease name confirmed case management means, it is possible to record and manage the disease name of the case of which the disease name has been confirmed, and the symptoms and the examination findings observed in the case.

Since the diagnosis support system includes the disease name unconfirmed case inquiry means, it is possible to, to the disease name confirmed case management means, present the symptoms and the examination findings of a disease name unconfirmed case and inquire about the disease name having high possibility and the symptoms and the examination findings to be obtained next that is effective for confirmation of the disease name.

Since the diagnosis support system according to claim 2 includes the case list management means for each symptom/finding, it is possible to manage a list of cases in which corresponding symptom/finding is observed, for each symptom/finding.

Since the diagnosis support system according to claim 3 includes the disease name list management means for each symptom/finding, it is possible to manage a list of each disease name in a corresponding case list, for each symptom/finding.

Since the diagnosis support system according to claim 4 includes the case list management means for each symptom/finding pair, it is possible to manage the case list for each symptom/finding pair observed simultaneously in each case.

Since the diagnosis support system according to claim 5 includes the case list management means for each symptom/finding for each disease name, it is possible to manage the list of observed cases for each symptom/finding observed in the case corresponding to the disease name.

Since the diagnosis support system according to claim 6 includes the case list management means for each symptom/finding pair for each disease name, it is possible to manage the case list for each symptom/finding pair simultaneously observed in each case corresponding to the disease name.

Since the diagnosis support system according to claim 7 includes the case list management means for each disease name, it is possible to manage a list of cases corresponding to the disease.

Since the diagnosis support system according to claim 8 includes the disease name confirmed case data management means, it is possible to record/manage a list of disease names and observed symptoms/findings of cases registered by the disease name confirmed case registration means.

Since the diagnosis support system according to claim 9 includes the similar case search means, it is possible to, for each of symptoms/findings observed in a disease name unconfirmed case, determine a product set of case lists having corresponding symptom/finding to display case data of corresponding case.

Since the diagnosis support system includes the similar case disease name estimation means, it is possible to aggregate the confirmed disease names of the cases obtained by the product set and display the confirmed disease names in order of frequency.

Since the diagnosis support system according to claim 10 includes the disease name candidate list estimation means, it is possible to estimate a disease name candidate list by using (i) frequency distribution of a confirmed disease name obtained by the disease name list management means for each symptom/finding for each symptom/finding observed in the disease name unconfirmed case, (ii) frequency distribution of the confirmed disease name of a case list obtained by the case list management means for each symptom/finding pair for each symptom/finding pair observed in the disease name unconfirmed case, or using both of (i) and (ii).

Since the diagnosis support system according to claim 11 includes the medical examination/test recommendation means, it is possible to compare aggregation frequencies of a case list for each symptom finding or symptom finding pair obtained by the case list management means for each symptom/finding for each disease name or the case list management means for each symptom/finding pair for each disease name, for each of the disease name candidates obtained by one of a confirmed disease name frequency order list obtained by the similar case search means or a disease name candidate list obtained by the disease name candidate list estimation means, to recommend a symptom/finding or symptom/finding pair with a large difference in aggregation frequency among the disease name candidates as a symptom/finding or symptom/finding pair to be obtained next.

Since the diagnosis support system according to claim 12 includes the recommendation degree reduction means using correlation, a symptom/finding that has not been performed and are highly correlated with the symptom/finding that has already been recommended and performed is determined by frequency distribution of confirmed disease names of the case list obtained by the case list management means for each symptom/finding pair to reduce a recommendation degree of the symptom/finding that has not been performed and are highly correlated.

Since the diagnosis support system according to claim 13 includes the cost corresponding medical examination and test recommendation means, a recommendation priority is adjusted by time and money costs required for execution.

Since the diagnosis support system according to claim 14 includes the symptom/finding case link group integration means, an inclusion relationship between individual symptoms/findings is described as a master-slave relationship link, and a case link group recorded in a slave symptom/finding is integrated to a master symptom/finding case link group as necessary.

Since the diagnosis support system according to claim 15 includes the disease name case link group integration means, an inclusion relationship between individual disease names is described as a master-slave relationship link, and a case link group recorded in a slave disease name is integrated to a master disease name case link group as necessary.

Since the diagnosis support system according to claim 16 includes the electronic medical record direct registration means, direct registration of a disease name and symptom/finding of a case of which disease name has been confirmed, from an electronic medical record of a patient is enabled.

Since the diagnosis support system according to claim 17 includes the electronic medical record direct inquiry means, direct inquiry of a symptom/finding of a case of which disease name has not been confirmed, from an electronic medical record of a patient to the disease name confirmed case management means is enabled.

Since the diagnosis support system according to claim 18 includes the case attribute management means, management is performed by imparting a case attribute such as a medical institution in which the case to be registered occurs, medical department, doctor in charge, and age, gender, and race of a patient.

Since the diagnosis support system according to claim 19 includes the search scope management means, only a case having a specific case attribute among the disease name confirmed cases managed by the disease name confirmed case management means is set as a search target.

Since the diagnosis support system according to claim 20 includes the disease name confirmed case exporting means, it is possible to export a part or all of contents recorded in the symptom/finding management means, the disease name management means, and the disease name confirmed case registration means included in the disease name confirmed case management means, to another disease name confirmed case management means.

Since the diagnosis support system according to claim 21 includes the disease name confirmed case importing means, it is possible to import the contents extracted by the disease name confirmed case exporting means to reconstruct the contents of the symptom/finding management means, the disease name management means, and the disease name confirmed case registration means.

Since the diagnosis support system according to claim 22 includes the disease name confirmed case management means content viewing means, it is possible to view a management content of the symptom/finding management means, the disease name management means, and the disease name confirmed case registration means.

### BRIEF DESCRIPTION OF DRAWINGS

FIG. 1 is a schematic diagram of a disease name confirmed case registration means, a disease name management means, and a symptom/finding management means included in a disease name confirmed case management means.

FIG. 2 is a schematic diagram in which a symptom/finding pair is also managed by the disease name confirmed case management means.

FIG. 3 is a diagram showing a list of cases corresponding to each symptom/finding in the symptom/finding management means.

FIG. 4 is a diagram showing a list of cases commonly included in each combination of symptom/finding and a list of cases commonly included in all symptoms/findings in FIG. 3.

FIG. 5 is a diagram showing a case list (product set) commonly included in all combinations of symptom/finding.

FIG. 6 is a diagram showing a list of cases that are commonly included in a combination of symptoms/findings even if some symptoms/findings are missing in FIG. 5.

FIG. 7 is a diagram showing the number of cases obtained by determining a confirmed disease name in a case list including all symptoms/findings commonly and totaling the number for each confirmed disease name in FIG. 5.

FIG. 8 is a diagram showing the total number of case lists for each symptom/finding or symptom/finding pair registered in the disease name management means for each disease name listed in a disease name candidate list.

FIG. 9 is a diagram showing a hierarchical structure of symptoms/findings.

FIG. 10 is a diagram showing a hierarchical structure of symptoms/findings, taking “chest pain” of symptoms/findings as an example, and showing links to master symptoms/findings and slave symptoms/findings.

### DESCRIPTION OF EMBODIMENTS

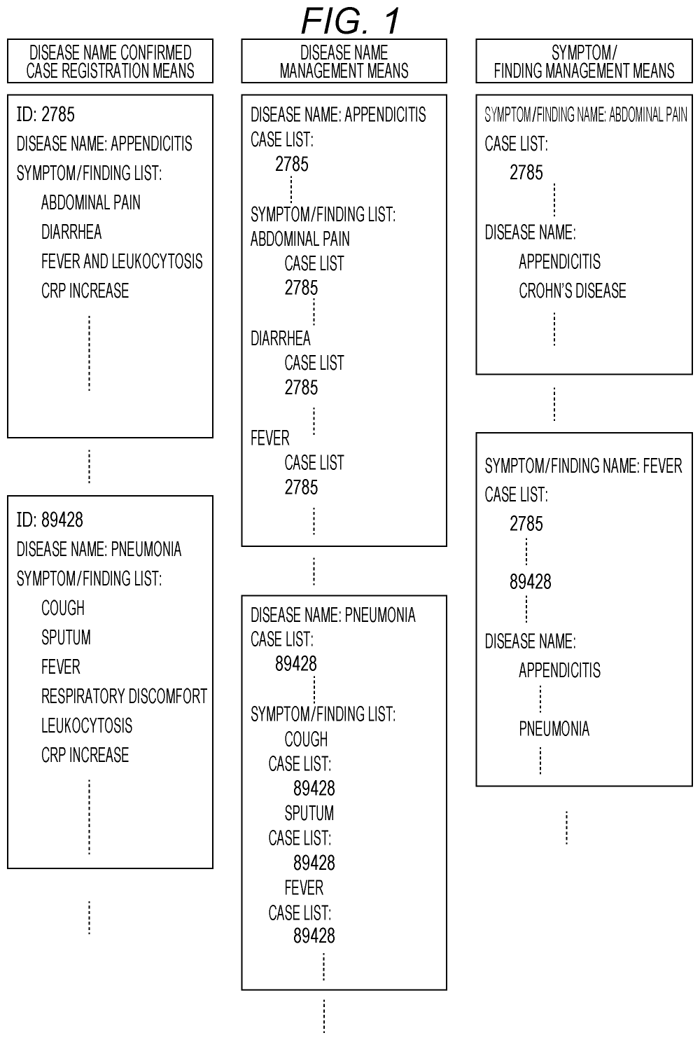

FIG. 1 is a schematic diagram of a disease name confirmed case registration means, a disease name management means, and a symptom/finding management means included in a disease name confirmed case management means of the present invention. The left column is a case data group including a patient ID of a case of which disease name has been confirmed, a disease name, and a list of symptoms/findings observed during the course.

Here, the case ID is for uniquely distinguishing each case, and may be numbered for each case registration by the disease name confirmed case registration means, or an electronic medical record of a medical institution may be enabled to be referred to using the medical institution ID+the patient ID at the medical institution.

When the same patient has a plurality of confirmed disease names, a branch number may be appropriately assigned to distinguish them.

Symptoms/findings are mainly cases where the symptoms/findings are positive, but negative symptoms/findings are often useful for differentiation of diseases. For this reason, negative findings may also be listed as independent symptoms/findings.

The case data may be discarded after being registered in the disease name management means and the symptom/finding management means, but it is desirable to manage the case data for each case ID for convenience of later reference (disease name confirmed case data management means).

If case data can be created from the summary data or the like of a case in an electronic medical record and directly passed to the disease name confirmed case registration means (electronic medical record direct registration means) every time the diagnosis name of the case is confirmed in daily medical care, it is desirable because there is no omission of registration of cases and the trouble of registration can be saved.

The central column of FIG. 1 is a schematic diagram of the disease name management means.

For each disease name, a list of cases corresponding to the disease name and a list of cases observed for each symptom/finding observed in the disease are managed.

For each case registered by the disease name confirmed case registration means, the case ID is registered in the case list of the disease name. By totaling the case IDs, the observation frequency of the case having the disease name can be known.

For each of the symptoms/findings of corresponding case, a case ID is registered in the case list of the corresponding symptoms/findings in the symptom/findings list of corresponding disease name. By totaling the case lists, the number of observation frequencies for each symptom/finding can be determined, and by dividing the number by the observation frequency of the disease name case, the observation rate for each symptom/finding can be determined.

Note that, although the list of cases corresponding to the disease name is created for the purpose of simple search, even by totaling the cases corresponding to the disease name each time by the disease name confirmed case registration means, it is possible to determine the observation frequency of the cases having the disease name.

The right column of FIG. 1 is a schematic diagram of the symptom/finding management means.

For each case registered by the disease name confirmed case registration means, the case ID is registered in the case list of corresponding symptom/finding for each symptom/finding observed in corresponding case.

Although a list of confirmed disease names for each case is created and recorded for the convenience of searching, but a list of confirmed disease names for each case ID may be created by JOIN operation of a relational database as needed.

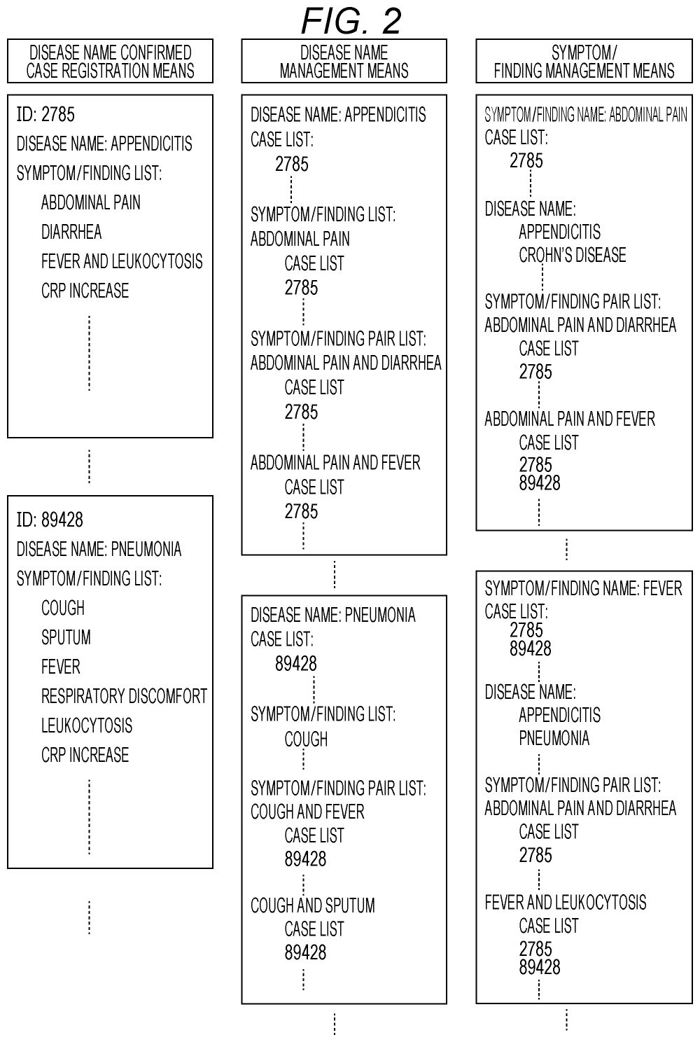

FIG. 2 is a schematic diagram in which a symptom/finding pair is also managed by the disease name confirmed case management means.

By also creating an observation case list for each symptom/finding pair, it is possible to grasp the observation rate in detail not only for the symptom/finding but also for the symptom/finding pair. In particular, it is possible to easily grasp the symptom/finding pairs that are correlated with each other.

Symptoms/findings are not necessarily independent and may correlate with each other. In this case, when one symptom/finding is observed, another symptom/finding is often observed or not observed simultaneously. As described above, even if the correlated symptoms/findings are duplicated, there is little new information that can be obtained. Accordingly, when the diagnosis is proceeded, it is required that correlated symptoms/findings are treated with lower priority.

With a conventional simple list of observed symptoms/findings, it is difficult to proceed with the above-mentioned differentiation, and it is essential to perform totaling of pairs of simultaneously observed symptoms/findings. Here, it is more useful to register a list of simultaneously observed symptoms/findings not only for positive findings but also for negative findings. Symptoms/findings that are highly correlated with each other often represent basically duplicate information, so that it is possible to lower the priority appropriately when estimating candidate disease names and when considering the next examination or test to be performed, which can reduce unnecessary medical examinations and tests.

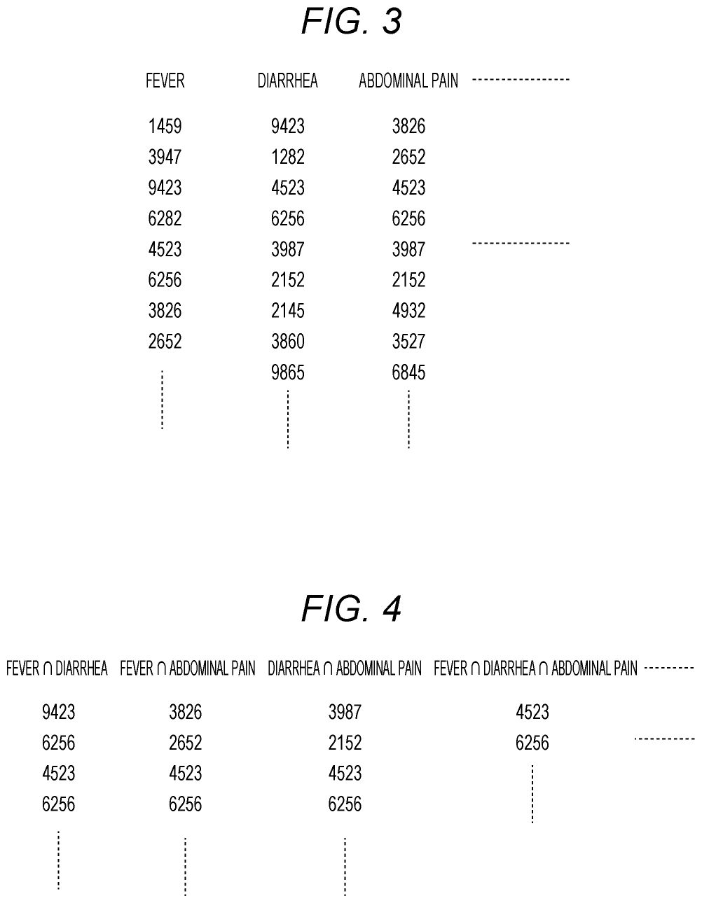

FIG. 3 is a diagram showing a list of cases corresponding to each of the symptoms/findings such as fever, diarrhea, and abdominal pain in the symptom/finding management means.

FIG. 4 shows a list (product set) of cases that are commonly included in each combination of symptoms/findings or that are commonly included in all symptoms/findings in FIG. 3.

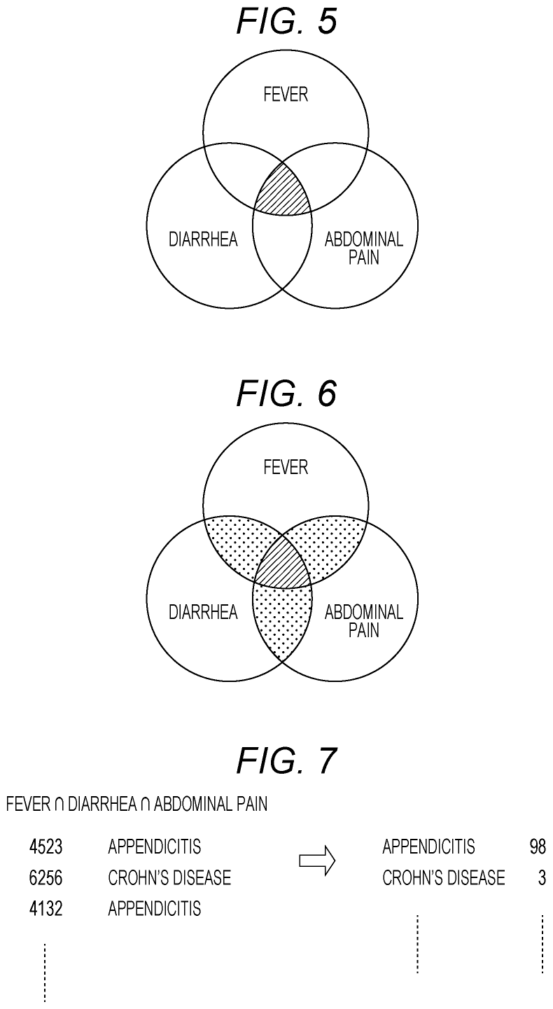

FIG. 5 is a schematic list of cases commonly included in all the above symptoms/findings.

As shown in FIG. 7, the disease name of each case can be determined from the list of these cases, the frequency can be totaled, and the disease name candidate can be displayed from the upper-ranked one.

As described above, by searching for past diagnosis name confirmed cases that are similar to a diagnosis name unconfirmed case on the basis of the symptoms/findings observed up to now in diagnosis name unconfirmed cases, totaling and arranging in frequency order the confirmed disease names of each case, a disease name candidate list of the disease name unconfirmed case can be obtained (similar case disease name estimation means).

In daily clinical practice, doctors often recall patients with similar symptoms/findings in the past and infer the diagnosis name. However, with the range of individual experience, the number of cases is limited, and the memory is ambiguous, so that the accuracy is also limited. Here, by listing the case data of the list of commonly included cases (similar case search means), it is possible to qualitatively grasp the observation pattern of the past diagnosis name confirmed case of the symptom/finding, and obtain suggestions for medical examinations and tests to be performed next.

If the number of lists of commonly included cases is too small, it may be a union of subsets of common cases with partial symptoms/findings, as shown in FIGS. 4 and 6.

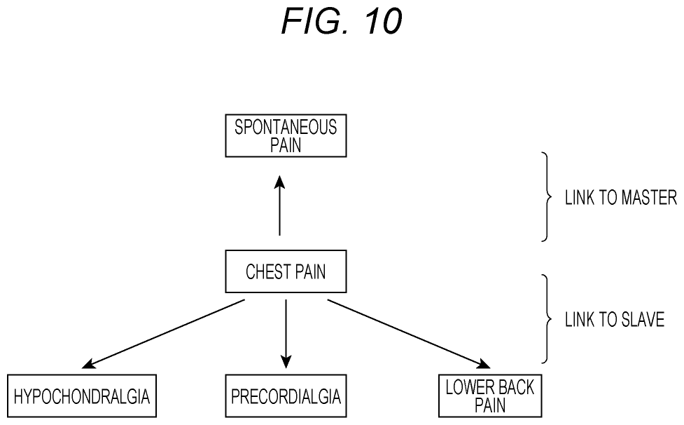

In addition, in the symptom/finding management means, as shown in FIGS. 9 and 10, by integrating case lists by using the symptom/finding case list group integration means that describes an inclusion relationship between individual symptoms/findings as a master-slave relationship link, and integrates a case list group recorded in a slave symptom/finding to a master case list group as necessary, it is possible to increase the number of the target case lists.

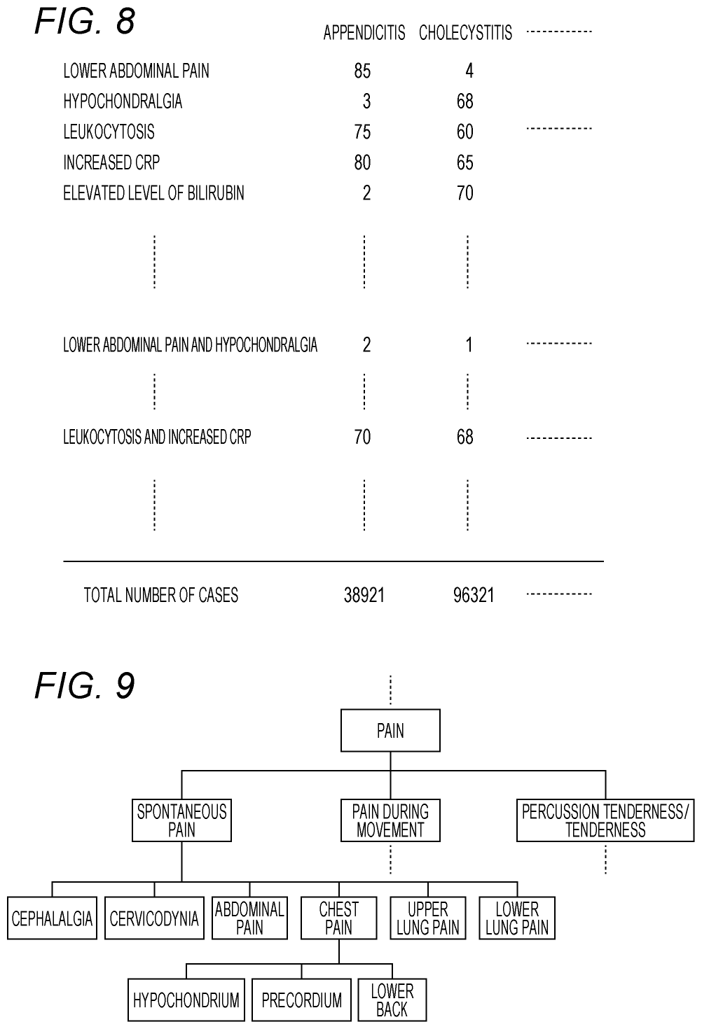

Another method for obtaining a disease name candidate list is a method of using a summary table of cases corresponding to a symptom/finding or symptom/finding pair for each disease name that is managed in the case list management means for each symptom/finding for each disease name or the case list management means for each symptom/finding pair for each disease name shown in FIG. 8. The frequency distribution of the disease names obtained by totaling corresponds to the prior probability of the disease name in Bayesian probability (in the drawing, 38921 cases of appendicitis, 96321 cases of cholecystitis, and the like are divided by the total number of cases). It is sufficient that each time a new symptom/finding is observed, the posterior probability of the disease name is updated (disease name list estimation means).

Here, if a plurality of candidates for the disease name is obtained, the next problem is what symptom/finding is to be obtained next in order to obtain a confirmed diagnosis.

Lower abdominal pain is more common in appendicitis and less common in cholecystitis. Conversely, hypochondralgia is less common in appendicitis and more common in cholecystitis. Accordingly, it can be seen that the symptoms of lower abdominal pain and hypochondralgia are useful for differentiation between appendicitis and cholecystitis. As described above, the symptom/finding or the symptom/finding pair having a large frequency difference between the disease names is useful for differentiation of the disease name.

On the other hand, the leukocytosis/CRP increase pair has a high frequency, and it can be seen that the two have a large correlation. Therefore, if one of these symptom/finding pairs is the next symptom/finding to be obtained, the other symptom/finding is likely to represent the same information, so that additional implementation only brings small amount of additional information. Accordingly, the priority of such symptoms/findings needs to be lowered.

Strictly speaking from a statistical point of view, it is necessary to obtain the partial correlation coefficient as a method of lowering the priority, but actual data is often missing and is not sufficient for analysis. In practice, conceivable is a method of dividing the symptom/finding pair frequency by the product of the square roots of each symptom/finding (pseudo-correlation coefficient), and multiplying the frequency of unimplemented symptoms/findings by (1-pseudo-correlation coefficient). An appropriate method may be selected in consideration of the usefulness and the time and effort required. In any case, some depreciation may be applied to the duplicated information.

In the drawing, for the sake of simplicity of explanation, two disease names, appendicitis and cholecystitis, are compared and examined, but in reality, it is not uncommon to compare and examine five to ten disease names. In some cases, a specific symptom/finding and a specific disease name correspond specifically to each other, but usually, the disease groups having the same tendency are gradually differentiated.

As described above, if the frequency distribution of symptoms and findings is obtained for each of the obtained disease name candidates, the disease name candidates are compared to each other, and the symptoms/findings with a larger frequency difference are more useful for confirmation of the diagnosis.

As mentioned above, it is sufficient that the priority of symptoms/findings that are likely to be observed simultaneously is lowered.

It is sufficient that medical examinations and tests to obtain the symptoms/findings are proceeded, and the posterior probabilities in the previous section are updated with the obtained observation results. The magnitude of the posterior probability that will change if the observation results are obtained is the degree of contribution to the confirmation of the diagnosis of the symptom/finding (medical examination and test recommendation method).

At this time, if the degree of contribution is the same, the one with less labor and cost is more practical.

It is sufficient that labor and costs are quantified, displayed in descending order of contribution to final confirmation of the diagnosis per cost, and then medical examinations and tests are proceeded (cost corresponding medical examination and test recommendation means). When the posterior probability reaches a certain level, the purpose is achieved.

In medical care, if a list of candidate disease names and recommendations for medical examinations and tests can be received while symptoms/findings in an electronic medical record are input, it is convenient for creating a plan for medical examinations and tests, and create a treatment plan according to the disease name (electronic medical record direct inquiry means).

It goes without saying that what is obtained here is probabilistic reasoning and is provided as a suggestion or assistance for a diagnosis by a doctor or the like, and is not a diagnosis itself with responsibility. That is, what is provided in the present invention is a candidate for a disease name, a probabilistic suggestion and recommendation of a medical examination or a test, and provides a judgment material to a doctor who is responsible for the judgment, not the judgment itself.

As described above, since the target diseases are different for each medical institution and each clinical department, the disease name and the frequency of observation of symptoms/findings are significantly different. For example, if there is a complaint of chest pain at a hospital specializing in heart disease, myocardial infarction is to be suspected first, but in orthopedics, there is a high possibility of a rib fracture.

As described above, the difference in frequency distribution depending on the attributes such as medical institutions and wards should be taken into consideration.

In addition, the frequency distribution differs depending on the attributes of the patient itself such as age, gender, and race.

It is useful that, in order to reflect the difference in frequency distribution due to such attributes in the inquiry results, the case attribute management means that adds medical care side attributes such as a medical institution, a medical department, or a doctor in charge, and patient attributes such as age, gender, or race to case data is included in the disease name confirmed case registration means, and the search scope management means that sets, as a search target, only case data having limited medical care side attributes and patient attributes is included in the disease name unconfirmed case inquiry means.

The diagnosis support system of the present invention is assumed to be constructed on the cloud.

However, some medical institutions operate on-premises electronic medical records without using connection to the Web for security reasons.

In such a case, by exporting a part or all of the management contents of the disease name confirmed case management means to another disease name confirmed case management means (disease name confirmed case exporting means), the case data can be shared with other medical institutions, and in some cases, it is possible to have the cost shared. By importing the exported contents as much as necessary for own hospital by the disease name confirmed case importing means, it is possible to share the case data according to the characteristics of the cases at own hospital.

With this function, it is possible to secure a sufficient amount of case data including that of other medical institutions in the disease name confirmed case management means without necessarily connecting to the Web at all times.

Including the disease name confirmed case management means content viewing means that develops the management contents of the symptom/finding management means, the disease name management means, and the disease name confirmed case registration means on a screen for viewing is useful for logical confirmation in finding inconsistency of management contents and proceeding of estimation of a disease name, medical examination, and test.

To implement the medical care support system of the present invention in a database, a graph database, a relational database (RDB) that has been widely used in the past, a key-value store (KVS) that has recently attracted attention as a method for processing big data and the like can be used. It is possible to use any implementation, but each has its own features/disadvantages.

First, the graph database is preferable in setting and displaying network-like graph relationships, but the processing speed is not fast in large-scale processing, so that it is not suitable for large-scale knowledge management systems. In RDB, it is sufficient that the registered contents are stored and managed in an XML object using a case ID, disease name, and symptom/finding name as keys.

Since the degree of freedom of search is high, the query language such as SQL is provided, and there are many software assets from the past, it is considered that implementation using the RDB is realistic.

The KVS includes one to several items of data (columns) and a set of key data. A wide variety of free searches are inferior to the RDB. However, for limited column data, high-speed distributed processing such as Map processing and Reduce processing is possible even for a large amount of data.

The RDB can also be used to determine the product set of long case lists, such as case lists, which can have hundreds of millions of data in one column, but there is a limit to work efficiency. When determining the frequency by multiple factors from large-scale case data, such as “How often are diabetic patients with HbA1c of greater than 10 and urinary protein of 2+ or higher?”, RDB's processing capacity is sometimes insufficient. In such cases, it is necessary to perform product set operation for the diabetic patient list, the HbA1c>10 patient list, and the urinary protein>2+ patient list. In such a case, distributed processing by KVS Map/Reduce is extremely effective.

As described above, it is considered that, in the medical care support system of the present invention, regardless of the database implementation method, a combination such as performing the basic part with the RDB and processing the case list and the like with the KVS is the most effective.

Although the embodiments have been described above, the specific configuration of the present invention is not limited to the above embodiments, and the present invention includes design changes and the like within a scope that does not deviate from the gist of the invention.

For example, the case data, disease name, symptom/finding name, observation frequency, and the like illustrated in the present application are merely examples for explanation. Those may be set appropriately according to the actual situation of medicine and medical care.

1. A diagnosis support system comprising a disease name confirmed case management means including
(i) a symptom/finding management means that records and manages a symptom and an examination finding of a patient whose diagnosis has been confirmed,
(ii) a disease name management means that records and manages, for each disease that has been confirmed, a list of cases corresponding to a corresponding disease, and a symptom and a finding observed in a corresponding case, and
(iii) a disease name confirmed case registration means that registers a disease name of a case of which disease name has been confirmed, and a symptom and a finding observed in a corresponding case in (i) and (ii),
wherein the diagnosis support system further includes a disease name unconfirmed case inquiry means that, to the disease name confirmed case management means, presents a symptom and an examination finding of a disease name unconfirmed case to inquire about a disease name having high possibility, a symptom and an examination finding to be obtained next which is effective for confirming a disease name.

2. The diagnosis support system according to claim 1, comprising, in the symptom/finding management means,
a case list management means for each symptom/finding that manages a list of cases in which a corresponding symptom/finding is observed for each symptom/finding.

3. The diagnosis support system according to claim 1, comprising, in the symptom/finding management means,
a disease name list management means for each symptom/finding that manages a list of disease names for each of a corresponding list of cases for each symptom/finding.

4. The diagnosis support system according to claim 2, comprising, in the case list management means for each symptom/finding,
a case list management means for each symptom/finding pair that manages a list of cases for each symptom/finding pair observed simultaneously in each case.

5. The diagnosis support system according to claim 1, comprising, in the disease name management means,
a case list management means for each symptom/finding for each disease name that manages a list of cases that have been observed for each symptom/finding that has been observed in cases corresponding to the disease name.

6. The diagnosis support system according to claim 5, comprising, in the case list management means for each symptom/finding for each disease name,
a case list management means for each symptom/finding pair for each disease name that manages a list of cases for each symptom/finding pair observed simultaneously in each case corresponding to a corresponding disease name.

7. The diagnosis support system according to claim 1, comprising, in the disease name management means,
a case list management means for each disease name that manages a list of cases corresponding to a corresponding disease.

8. The diagnosis support system according to claim 1, comprising, in the disease name confirmed case management means,
a disease name confirmed case data management means that records/manages a list of a disease name of a case registered in the disease name confirmed case registration means and a symptom/finding that has been observed.

9. The diagnosis support system according to claim 1, comprising, in the disease name unconfirmed case inquiry means,
at least one of: a similar case search means that obtains, for each symptom/finding observed in the disease name unconfirmed case, a product set of a list of cases having a corresponding symptom/finding to display case data of corresponding case; and a similar case disease name estimation means that totals confirmed disease names of cases obtained by the product set to display the confirmed disease names in order of frequency.

10. The diagnosis support system according to claim 1, comprising, in the disease name unconfirmed case inquiry means,
a disease name candidate list estimation means that estimates a disease name candidate list by using (i) frequency distribution of a confirmed disease name obtained by the disease name list management means for each symptom/finding for each symptom/finding, the frequency distribution being observed in a corresponding disease name unconfirmed case, (ii) frequency distribution of a confirmed disease name of a case list obtained by the case list management means for each symptom/finding pair for each symptom/finding pair, the frequency distribution being observed in a corresponding disease name unconfirmed case, or using both of (i) and (ii).

11. The diagnosis support system according to claim 10, comprising, in the disease name unconfirmed case inquiry means,
a medical examination/test recommendation means that compares aggregation frequencies of a case list for each symptom finding or symptom finding pair obtained by the case list management means for each symptom/finding for each disease name or the case list management means for each symptom/finding pair for each disease name, for each of disease name candidates obtained by one of a confirmed disease name frequency order list obtained by the similar case disease name estimation means or a disease name candidate list obtained by the disease name candidate list estimation means, to recommend a symptom/finding or symptom/finding pair with a large difference in aggregation frequency among the disease name candidates as a symptom/finding or symptom/finding pair to be obtained next.

12. The diagnosis support system according to claim 11, comprising, in the medical examination/test recommendation means,
a recommendation degree reduction means, using correlation, that determines a symptom/finding that has not been performed and are highly correlated with a symptom/finding that has already been recommended and performed by frequency distribution of confirmed disease names of a case list obtained by the case list management means for each symptom/finding pair to reduce a recommendation degree of the symptom/finding that has not been performed and are highly correlated.

13. The diagnosis support system according to claim 11, comprising, in the medical examination/test recommendation means,
a cost corresponding medical examination and test recommendation means that adjusts a recommendation priority by time and money costs required for execution.

14. The diagnosis support system according to claim 1, comprising, in the symptom/finding management means,
a symptom/finding case link group integration means that describes an inclusion relationship between individual symptom/finding as a master-slave relationship link, and integrates a case link group recorded in a slave symptom/finding to a master case link group as necessary.

15. The diagnosis support system according to claim 1, comprising, in the disease name management means,
a disease name case link group integration means that describes an inclusion relationship between individual disease names as a master-slave relationship link, and integrates a case link group recorded in a slave disease name to a master disease name case link group as necessary.

16. The diagnosis support system according to claim 1, comprising, in the disease name confirmed case registration means,
an electronic medical record direct registration means that enables direct registration of a disease name and symptom/finding of a case of which disease name has been confirmed, from an electronic medical record of a corresponding patient.

17. The diagnosis support system according to claim 1, comprising, in the disease name unconfirmed case inquiry means,
an electronic medical record direct inquiry means that enables direct inquiry of a symptom/finding of a case of which disease name has not been confirmed, from an electronic medical record of a corresponding patient to the disease name confirmed case management means.

18. The diagnosis support system according to claim 1, comprising, in the disease name confirmed case registration means,
a case attribute management means that performs management by imparting a case attribute such as a medical institution in which a case to be registered occurs, medical department, doctor in charge, and age, gender, and race of a patient.

19. The diagnosis support system according to claim 1, comprising, in the disease name unconfirmed case inquiry means,
a search scope management means that sets, as a search target, only a case having a specific case attribute among disease name confirmed cases managed by the disease name confirmed case management means.

20. The diagnosis support system according to claim 1, comprising a disease name confirmed case exporting means that exports a part or all of contents recorded in the symptom/finding management means, the disease name management means, and the disease name confirmed case registration means included in the disease name confirmed case management means, to another disease name confirmed case management means.

21. The diagnosis support system according to claim 20, comprising a disease name confirmed case importing means that imports contents extracted by the disease name confirmed case exporting means to reconstruct contents of the symptom/finding management means, the disease name management means, and the disease name confirmed case registration means.

22. The diagnosis support system according to claim 1, comprising, in the disease name confirmed case management means,
a disease name confirmed case management means content viewing means that views a management content of the symptom/finding management means, the disease name management means, and the disease name confirmed case registration means.
